(19) United States
(12) Patent Application Publication
(10) Pub. No.: US 2013/0116786 A1
(43) Pub. Date: May 9, 2013

(54) BREAST PROSTHESIS FILLED WITH MICROSPHERES OF THERMOEXPANDED POLYMER

(71) Applicant: FREDDY SANABRIA SCHARF, BOGOTA (CO)

(72) Inventor: FREDDY SANABRIA SCHARF, BOGOTA (CO)

(21) Appl. No.: 13/648,928

(22) Filed: Oct. 10, 2012

(30) Foreign Application Priority Data
Nov. 4, 2011 (CO) 11-150025

(51) Int. Cl. A61F 2/12

(52) U.S. Cl. CPC A61F 2/12
USPC 623/8

(57) ABSTRACT

A breast prosthesis constructed from a bag of elastic material or biocompatible mesh having a front face forming a dome and a rear face forming a base with a sealed and seamless interior cavity filled with microspheres of foam polymers or thereto expanded polymers or a combination of both.

### SUMMARY WITH THE OBJECTIVE AND PURPOSE OF THE UTILITY MODEL

This invention is generally related to the breast implant with thermo-expanded polymer microspheres filler. Concretely, the invention consists of a breast implant comprising an implant pocket or shell with an interior cavity, based on elastic material, and which has a front part, that constitutes a dome, a back part that constitutes a base, a shaped pocket that resembles a full and round female breast, of watertight form, and that includes a thermo-expanded polymer microspheres filler, where the microspheres can be enclosed in a silicone pocket (shell) or in a mesh-like pocket made of biocompatible material.

The implant, object of the invention, can present an alternative to the already existent implants with silicone or saline solution filler, and offers advantage to the prior art: it avoids loss of volume, it can be an alternative for the weight loss, it facilitates the removal of the mammary pouch and diminish the consequences of rupture inside of the mammary pouch, such as siliconomas and seromas formation. As an alternative it can improve resistance and more natural sensation to touch when implanted.

### DESCRIPTION OF THE UTILITY MODEL

### Prior Art

This invention is related in general to the breast implant with thermo-expanded polymer microspheres filler. This invention implant presents an alternative to the already existent implants with silicone gel or saline solution filler, and offers advantage to the existing implants: it can diminish the consequences of rupture inside of the mammary pouch, such as siliconomas and seromas formation, it makes more natural the sensation to touch more natural once implanted; weight loss; facilitates the withdrawal of the mammary pouch.

The breast implants are intended for the esthetic breast augmentation and for reconstruction after an ablation by surgery as an oncologic or traumatologic treatment. This surgical definition originates in the field of reconstructive and plastic surgery. The development concerning breast implants can be dated since more than 30 years ago. Research into resistant elastic coatings with long-term reliability has been conducted, such as the use of silicone gel fillers of high cohesiveness and saline solution and soy oil (trilucent) fillers.

Due to technical development and a vast market for the above mentioned product it is being pondered to start the development of implants for other parts of the body, like breast implants, the glutes or testicles among others, in which the filler would not be a liquid or a gel, or their combination, and which diminishes the complications implied by the actual implants, such as gel deflation and migration that can generate seromas, siliconomas and lympadenopathy, consequences of rupture inside the mammary cavity and replacement of a broken implant.

As indicated, the prior art in the field of implants presents various disadvantages that the proposed invention intends to overcome or diminish, establishing itself as an alternative to the already existent. In comparison to the generality of the implants, the proposed invention presents advantages in case of rupture, avoiding the complications of the existent implants. In the case of saline solution filled implants, it has been observed that, once implanted, these lose volume in the course of time and they have a sensation to touch far from natural, which the proposed invention intends to overcome. Concerning the silicone filler implants, the viscosity and density of the gel can lead to implants of higher weight and in the case of eventual rupture, the gel spilling and migration inside the mammary pouch can complicate to the doctor its removal, generate seromas or siliconomas, which effects can be mitigated by the filler of the proposed invention as it allows to remove the filler easier before eventual rupture. Additionally, the characteristics of the implant, object of the invention, allow to remove the implant easier and improve the resistance due to its characteristic thermo-expanded polymer microspheres filler composition; less weight of the implant and more natural sensation to touch once implanted.

The breast augmentation using implants should be realized in a surgical environment (operating theater) by surgeons specialized in plastic surgery. Among the surgical techniques to realize this type of surgery, the surgeon should make incision according to the place of the scar. The most widespread incision type is periareolar (along the periphery of the areola). The second manner is the transaxillary incision (made to the axilla) and the third one generally used is the inframammary insicion made to the inframammary fold.

The implant should be emplaced to one of the two available places of the human body: subglandular, between the mammary gland and the pectoralis major muscle, beneath the mammary gland and the pectoralis major muscle. It is the surgeon's responsibility to determine the size of the implant, the incision site on the chest, the approach to the surgical site and the criteria for the emplacement of the implant considering the anatomy of the patient and the desired esthetic result.

Some combined systems that include a gel with silicone microspheres have been developed. This development is related in the U.S. Pat. No. 4,380,569 Shaw from 19 Apr. 1983, as a breast implant with reduced weight consisting of a mixture of a gel with silicone microspheres dispersed in the liquid. In this proposal, the weight is reduced but the problem of the liquid pressure, friction and gel presence, with the already mentioned disadvantages, persists.

This being the state of affairs, the implant, object of the invention, is intended to be an alternative solution to the generality of implants with silicone gel fillers, saline solution fillers and their combinations. It constitutes an innovating implant with a filler consisting of microspheres of a thermo-expandible and biocompatible material, inside of a pocket made of silicone or other biocompatible material. It is an implant that can be a solution or mitigation of the problems that the presently used implants cause.

### OBJECT OF THE INVENTION

Accordingly, a first object of the present invention is to avoid the disadvantages and complications of the prior art, and constitute a possible alternative. More particularly, an object of the present invention is an improvement to the prior art, where the breast implant will comprise a bag or pocket receiver that resembles the breast, the material may be silicone, or any other biocompatible material which will fill solely and exclusively biocompatible polymer microspheres, where the entire implant bag and filler, results in an implant that avoids or mitigates the complications of the prior art. In particular, resulting in an implant which mitigates volume loss as saline, and the consequences of a possible rupture of the implant within the breast cavity of silicone gel, allowing the filler to be removed more easily, in contrast with what happens with a material such as silicone gel. Additionally, the stability of the filler against liquids and gels allows it to be more easily removed from the breast pocket against the existing art that is of lighter weight than the prior art, and a potential strength and durability.

The present invention fulfills these needs and provides other related advantages.

The novel features which are considered as the basis for the invention are set forth particularly in the appended claims and the advantages thereof, it will be best understood on the following detailed description of preferred embodiments and with due reference to the accompanying drawings.

### BRIEF DESCRIPTION OF THE FIGURES

To further clarify the invention and its advantages compared with the known art, they are described below with the aid of the annexed drawings, possible forms of non-limiting exemplary embodiments and application of those principles.

FIG. 1 shows an isometric view of a breast implant with a cut on the surface of the present invention in its preferred embodiment.

### DETAILED DESCRIPTION OF THE INVENTION

This invention relates generally with breast prosthesis with microsphere filter.

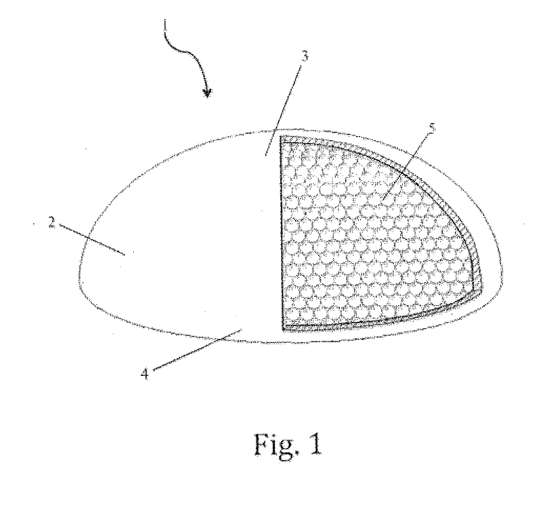

Particularly based on FIG. 1, the invention comprises 1 breast prosthesis constructed from a bag or envelope with internal cavity, based on a resilient material such as silicon elastomer, or any other material, biocompatible, that fulfills the function of bag, mesh or receptor of microspheres. Conveniently the enclosure material may comprise a bag made from a silicone elastomer or a bag, mesh, biocompatible.

This being the case, the prosthesis 1 comprises a bag 2 formed from a front face which forms a dome 3 and a rear face forming the base 4, where said bag 2 is of configuration similar to a female breast.

This bag 2 is filled with microspheres. The microspheres 5 may be contained in a bag of silicon or a bag type mesh of biocompatible material. Such microspheres are molded from foam polymers or term expanded such as ePTFE polytetrafluoroethylene or polystyrene or a combination of various materials.

Optionally, said microspheres may comprise a core made from a material, coated with a different material, as long as said materials comprise a selection of biocompatible material.

As such, the bag is closed, seamless, sealingly to the environment in such a manner that the microspheres do not make contact with the breast cavity.

As such, the implant of the present invention includes various sizes to balance bust-shaped differences, giving the surgeon a wider range of options; these configurations are available with a smooth or textured surface.

For the needs for manufacturing of the bag or mesh, the elastic material suitable for this purpose, is the silicon, but may also be any biocompatible material that fulfills the function of bag or receiver of the microspheres. An indication can understand that material manufactured and marketed by NUSIL Technology® (ISO 9001 certificate).

They have only been illustrated as example of some preferred embodiments of invention. In this regard, it will be appreciated that the construction of the breast implant, as well as the arrangements in its particular configuration may be selected from a plurality of alternatives without departing from the spirit of the invention according to the following claims.

1. (canceled)

2. (canceled)

3. (canceled)

4. (canceled)

5. A breast prosthesis constructed from a bag having a sealed and seamless interior cavity, where the bag has a front face that forms a dome and a rear face that forms a base, and where the interior cavity contains a plurality of microspheres.

6. A breast prosthesis according to claim 5 where the microspheres are made from foam polymers.

7. A breast prosthesis according to claim 5 where the microspheres are made from thermo expanded polymers.

8. A breast prosthesis according to claim 5 where the microspheres are made from a combination of foam and thermo expanded polymers.

9. A breast prosthesis according to claim 6 where the bag is made of elastic material.

10. A breast prosthesis according to claim 9 where the elastic material is silicone elastomer.

11. A breast prosthesis according to claim 7 where the bag is made of elastic material.

12. A breast prosthesis according to claim 11 where the elastic material is silicone elastomer.

13. A breast prosthesis according to claim 8 where the bag is made of elastic material.

14. A breast prosthesis according to claim 13 where the elastic material is silicone elastomer.

15. A breast prosthesis according to claim 6 where the bag is a mesh of biocompatible material.

16. A breast prosthesis according to claim 7 where the bag is a mesh of biocompatible material.

17. A breast prosthesis according to claim 8 where the bag is a mesh of biocompatible material.
